(19) United States
(12) Patent Application Publication
(10) Pub. No.: US 2026/0063586 A1
(43) Pub. Date: Mar. 5, 2026

(54) NANOSEQUENCING DEVICE BASED ON REDOX-LABELING AND A ROTATIONALLY SYMMETRIC ELECTRODE ARRANGEMENT AROUND A NANOPORE

(71) Applicant: Robert Bosch GmbH, Stuttgart (DE)

(72) Inventors: Franz LAERMER, WEIL DER STADT (DE); Martina HUEBNER, RENNINGEN (DE); Nadezda FOMINA, REDWOOD CITY, CA (US); Christopher JOHNSON, SAN CARLOS, CA (US); Shin YOUNG SHIK, MOUNTAIN VIEW, CA (US); Christoph LANG, SUNNYVALE, CA (US)

(21) Appl. No.: 18/820,896

(22) Filed: Aug. 30, 2024

(51) Int. Cl. G01N 27/327; C09K 13/00; C12Q 1/6869

(52) U.S. Cl. CPC G01N 27/3278; C09K 13/00; C12Q 1/6869; G01N 27/3277

(57) ABSTRACT

A system for sequencing a polynucleotide strand including a device with a layer stack on a silicon wafer. The layer stack may include a first electrode and a second electrode separated by a dielectric insulating layer and a nanohole drilled through the layer stack so that the first and second electrodes are rotationally arranged around the nanohole. A polynucleotide strand with at least one nucleotide and at least one electroactive label may be pulled through the nanohole. The system may additionally include a controller that may apply an electrical signal to the first and second electrodes, measure a current (I) as a function of an applied potential (V) when an electroactive label is guided through the nanohole of the device, and adjust an electrical voltage to pull the polynucleotide strand through the nanohole at a predetermined speed.

### TECHNICAL FIELD

The present disclosure relates to systems and methods for nanopore sequencing.

### BACKGROUND

Next-generation sequencing solutions have included guiding polynucleotides or polypeptides through solid-state nanostructures including solid-state nanopores or nanochannels for example. Resolution depends critically on the thickness of the membrane bearing the nanopore which can limit the choice of suitable materials to exotic candidates and also affect the mechanical stability of the membrane construction.

Ultrathin lateral electrodes split into two symmetric half-shells opposing each other around the nanopore have also been investigated. When a nucleotide is passing through a nanopore in between the split two opposing half-shells electrodes on each side, the current between the lateral electrodes is modulated. These systems however exhibit lower than desirable resolution performance and are technically difficult to synthesize.

Nanochannels equipped with integrated comb electrodes (with dielectric nanogaps insulating the comb electrodes from each other) have also been utilized. These systems, however, require a highly complex manufacturing process. Additionally, nucleotide detection depends strongly on the distance and orientation of labeled DNA with respect to the nanogap electrodes. Orientation dependance can lead to information loss in that only events that are properly oriented will be discovered. As such, there is a need for methods and systems for nanopore sequencing that overcome the aforementioned limitations and offer additional solutions.

### SUMMARY

In at least an aspect, a system for sequencing a polynucleotide strand is provided. The system may comprise a device including a layer stack on a silicon wafer, the layer stack comprising a first electrode and a second electrode separated by a dielectric insulating layer. The device may also include a nanohole drilled through the layer stack so that the first and second electrodes are rotationally arranged around the nanohole, wherein the nanohole is sized to receive a polynucleotide strand with at least one nucleotide and at least one electroactive label. The system may additionally include a controller configured to apply an electrical signal to the first and second electrodes; measure a current (I) as a function of an applied potential (V) when an electroactive label is guided through the nanohole of the device; and adjust an electrical voltage to pull the polynucleotide strand through the nanohole at a predetermined speed.

In at least another aspect, a method for sequencing a polynucleotide strand is provided. The method may comprise providing a sample containing a polynucleotide strand with a first nucleotide having a first electroactive label and a second nucleotide having a second electroactive label. The second electroactive label may be distinguishable from the first electroactive label. The sample may be provided to a device including a layer stack on a silicon wafer. The layer stack may comprise a first electrode and a second electrode separated by a dielectric insulating layer, and a nanohole drilled through the layer stack so that the first and second electrodes are rotationally arranged around the nanohole. The method may additionally comprise applying an electrical signal to the first and second electrodes and measuring a current (I) as a function of an applied potential (V) when the first and the second electroactive labels are present in the nanohole.

In yet another aspect, a method for forming a system for sequencing a polynucleotide strand is provided. The method may comprise assembling a base wafer by forming an etching mask on a backside of a silicon wafer, an etching stop on an opposite side of the silicon wafer, and a layer stack comprising a first electrode and a second electrode separated by a dielectric insulating layer on the etching stop. The method may further comprise backside etching through the base wafer; removing the backside etching mask; opening a front side window; and forming a nanohole through the layer stack. The first and second electrodes may be rotationally arranged around the nanohole to form a device. The method may also comprise configuring a controller to apply an electrical signal to the first and second electrodes, and to measure a current (I) as a function of an applied potential (V) when an electroactive label is present in the nanohole.

### BRIEF DESCRIPTION OF THE DRAWINGS

FIG. 1 illustrates a layer stack on a base wafer according to an embodiment.

FIG. 2 illustrates a cross section of the wafer after backside etching through the wafer according to an embodiment.

FIG. 3 illustrates the removal of the etch-stop layer underneath the metal-oxide-metal electrode stack according to an embodiment.

FIG. 4 illustrates a schematic of a completed device with a nanohole drilled through the membrane according to an embodiment.

FIG. 5 illustrates a cross-section view of redox-labeled DNA inside a nanopore in-between electrodes on the membrane according to an embodiment.

FIG. 6 illustrates a top view of redox-labeled DNA inside a nanopore in-between electrodes on the membrane according to an embodiment.

FIG. 7 illustrates a device having a three-electrode arrangement according to an embodiment.

FIG. 8 illustrates a schematic of an electrode arrangement that includes two electrodes separated by a nanogap, with each pair of electrodes being separated from the next by an insulating layer.

FIG. 9 illustrates a schematic of an electrode arrangement that includes three electrodes separated by two identical nanogaps. Each group of electrodes is separated from the next group by an insulating layer.

FIG. 10 illustrates cyclic voltammograms of electroactive labels in 10 mM Phosphate buffer solution containing 100 mM KCl and 10% DMSO.

### DETAILED DESCRIPTION

As required, detailed embodiments of the present disclosure are disclosed herein; however, it is to be understood that the disclosed embodiments are merely exemplary of the disclosure that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present disclosure.

Except in the examples, or where otherwise expressly indicated, all numerical quantities in this description indicating amounts of material or conditions of reaction and/or use are to be understood as modified by the word “about”. The first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

Unless indicated otherwise, all technical and scientific terms used herein have the same meaning as commonly understood by one of ordinary skill in the art to which the present disclosure belongs.

It is also to be understood that this disclosure is not limited to the specific embodiments and methods described below, as specific components and/or conditions may, of course, vary. Furthermore, the terminology used herein is used only for describing particular embodiments and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The terms “or” and “and” can be used interchangeably and can be understood to mean “and/or”.

The term “comprising” is synonymous with “including,” “having,” “containing,” or “characterized by. ” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

The terms “comprising”, “consisting of”, and “consisting essentially of” can be alternatively used. When one of these three terms is used, the presently disclosed and claimed subject matter can include the use of either of the other two terms.

The terms “polynucleotide”, “polynucleotide strand”, “nucleotide”, “nucleotide sequence”, “nucleic acid” and “oligonucleotide” may be used interchangeably in this disclosure. They may refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The terms “polynucleotide” and “nucleic acid” should be understood to include, as applicable to the embodiment being described, single-stranded (such as sense or antisense) and double-stranded polynucleotides. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The terms “complementarity” or “complement” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 4, 5, and 6 out of 6 being 66.67%, 83.33%, and 100% complementary). “Perfectly complementary” may refer to all the contiguous residues of a nucleic acid sequence may hydrogen bond with the same number of contiguous residues in a second nucleic acid sequence. “Substantially complementary” as used herein may refer to a degree of complementarity that is at least 40%, 50%, 60%, 62.5%, 70%, 75%, 80%, 85%, 90%, 95%, 97%, 98%, 99%, or 100%, or percentages in between over a region of 4, 5, 6, 7, and 8 nucleotides, or refers to two nucleic acids that hybridize under stringent conditions.

The term “enzyme” or “polymerase” as used herein may refer to any peptide, oligopeptide, polypeptide, gene product, expression product, or protein capable of translocating a polynucleotide strand. Non-limiting examples of proteins capable of translocating a polynucleotide strand include DNA polymerase, RNA polymerase, ribosome, a single-stranded binding protein, topoisomerase, helicase, nuclease, exonuclease, endonuclease, a zinc finger nuclease, an RNA guided DNA endonuclease, a transcription activator-like effector nuclease, a CRISPR protein, and combinations thereof.

Unless expressly stated to the contrary, all R groups (e.g. Rᵢ where i is an integer) include hydrogen, alkyl, lower alkyl, C₁₋₆ alkyl, C₆₋₁₀ aryl, C₆₋₁₀ heteroaryl, —NO₂, —NH₂, —N(R′R″)₂, —N(R′R″R″′)₃⁺L⁻, Cl, F, Br, —CF₃, —CCl₃, —CN, —SO₃H, —PO₃H₂, —COOH, —CO₂R′. —COR′, —CHO, —OH, —OR′, —O⁻M⁺, —SO₃⁻M⁺, —PO₃⁻M⁺, —COO⁻M⁺, —CF₂H, —CF₂H, —CF₂R′, —CFH₂, and —CFR′R″ where R′, R″ and R″′ are C₁₋₁₀ alkyl or C₆₋₁₈ aryl groups; single letters (e.g., “n” or “o”) are 1, 2, 3, 4, or 5; in the compounds disclosed herein a CH bond can be substituted with alkyl, lower alkyl, C₁₋₆ alkyl, C₆₋₁₀ aryl, C₆₋₁₀ heteroaryl, —NO₂, —NH₂, —N(R′R″)₂, —N(R′R″R″′)₃⁺L⁻, Cl, F, Br, —CF₃, —CCL₃, —CN, —SO₃H, —PO₃H₂, —COOH, —CO₂R′, —COR′, —CHO, —OH, —OR′, —O⁻M⁺, —SO₃⁻M⁺, —PO₃M⁺, —COO⁻M⁺, —CF₂H, —CF₂R′, —CFH₂, and —CFR′R″ where R′, R″ and R″′ are C₁₋₁₀ alkyl or C₆₋₁₈ aryl groups; the indication of a moiety or structure with positive charges implies that one or more negative counter ions are present to balance the charge, similarly, the indication of a moiety or structure with negative charges implies that one or more positive counter ions are present to balance the charge; percent, “parts of,” and ratio values are by weight; the term “polymer” includes “oligomer,” “copolymer,” “terpolymer,” and the like; molecular weights provided for any polymers may refer to weight average molecular weight unless otherwise indicated; the description of a group or class of materials as suitable for a given purpose in connection with one or more embodiments may imply that mixtures of any two or more of the members of the group or class are equally suitable; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

The term “alkyl” as used herein may mean C₁₋₂₀, linear, branched, rings, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Lower alkyl” refers to an alkyl group having 1 to about 8 carbon atoms (i.e., a C₁₋₈alkyl), e.g., 1, 2, 3, 4, 5, 6, 7, or 8 carbon atoms. Lower alkyl may also refer to a range between any two numbers of carbon atoms listed above. “Higher alkyl” refers to an alkyl group having about 10 to about 20 carbon atoms, e.g., 10, 11, 12, 13, 14, 15, 16, 17, 18, 19, or 20 carbon atoms. Higher alkyl may also refer to a range between any two number of carbon atoms listed above.

The term “aryl” as used herein may mean an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether. Examples of aryl include, but are not limited to, phenyl, naphthyl, biphenyl, and diphenylether, and the like. Aryl groups include heteroaryl groups, wherein the aromatic ring or rings include a heteroatom (e.g., N, O, S, or Se). Exemplary heteroaryl groups include, but are not limited to, furanyl, pyridyl, pyrimidinyl, imidazoyl, benzimidazolyl, benzofuranyl, benzothiophenyl, quinolinyl, isoquinolinyl, thiophenyl, and the like. The aryl group may be optionally substituted (a “substituted aryl”) with one or more aryl group substituents, which can be the same or different, wherein “aryl group substituent” includes alkyl (saturated or unsaturated), substituted alkyl (e.g., haloalkyl and perhaloalkyl, such as but not limited to —CF₃), ycloalkyl, aryl, substituted aryl, aralkyl, halo, nitro, hydroxyl, acyl, carboxyl, alkoxyl (e.g., methoxy), aryloxyl, aralkyloxyl, thioalkyl, thioaryl, thioaralkyl, amino (e.g., aminoalkyl, aminodialkyl, aminoaryl, etc.), sulfonyl, and sulfinyl.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits. In the specific examples set forth herein, concentrations, temperature, and reaction conditions (e.g., pressure, pH, etc.) can be practiced with plus or minus 50 percent of the values indicated rounded to three significant figures. In a refinement, concentrations, temperature, and reaction conditions (e.g., pressure, pH, etc.) can be practiced with plus or minus 30 percent of the values indicated rounded to three significant figures of the value provided in the examples. In another refinement, concentrations, temperature, and reaction conditions (e.g., pH, etc.) can be practiced with plus or minus 10 percent of the values indicated rounded to three significant figures of the value provided in the examples.

In the examples set forth herein, concentrations, temperature, and reaction conditions (e.g., pressure, pH, flow rates, etc.) can be practiced with plus or minus 50 percent of the values indicated rounded to or truncated to two significant figures of the value provided in the examples. In a refinement, concentrations, temperature, and reaction conditions (e.g., pressure, pH, flow rates, etc.) can be practiced with plus or minus 30 percent of the values indicated rounded to or truncated to two significant figures of the value provided in the examples. In another refinement, concentrations, temperature, and reaction conditions (e.g., pressure, pH, flow rates, etc.) can be practiced with plus or minus 10 percent of the values indicated rounded to or truncated to two significant figures of the value provided in the examples.

In this application, electroactive molecules include Redox molecules. A Redox signal includes electrical signals such as a change in baseline current that may be visualized in the form of spikes or fluctuations for example. Polynucleic acid (NA), or polynucleotide strand includes DNA or RNA, and nucleotides include dNTPs.

The processes, methods, or algorithms disclosed herein can be deliverable to/implemented by a processing device, controller, or computer, which can include any existing programmable electronic control unit or dedicated electronic control unit. Similarly, the processes, methods, or algorithms can be stored as data and instructions executable by a controller or computer in many forms. The processes, methods, or algorithms can also be implemented in an executable software object. Alternatively, the processes, methods, or algorithms can be embodied in whole or in part using suitable hardware components, such as Application Specific Integrated Circuits (ASICs), Field-Programmable Gate Arrays (FPGAs), state machines, controllers or other hardware components or devices, or a combination of hardware, software, and firmware components.

Throughout this application, where publications are referenced, the disclosures of these publications in their entireties are hereby incorporated by reference into this application to more fully describe the state of the art to which this disclosure pertains.

Provided herein are next-generation sequencing solutions. The systems and methods described in this disclosure include systems and methods involving guiding polynucleotides including DNA or RNA, or polypeptides including proteins through solid-state nanostructures. For example, the systems and methods may involve guiding polynucleotides or polypeptides through solid-state nanopores or nanochannels.

Solid-state nanopores have been used for sequencing purposes. DNA for example, may be pulled through a nanopore perforating a thin membrane, while the electrical current flowing through the nanopore at the same time and same direction is measured. The electrical current is modulated by the nucleotides which are partially blocking the nanopore during passage sequentially one by one. Variations in current caused by the nucleotides passaging through the nanopore are extremely small, far below 1 pA, or even far below 100 fA. The electrical voltage used to generate the current is limited in size since it also affects the speed of DNA passage. There is a tradeoff between the desired voltage level (as high as possible for higher electrical signals) and low speed of DNA in the nanopore (voltage and speed as small as possible for enhanced resolution and lower bandwidth needs). A significant limitation of current nanopore solutions is that the resolution depends critically on the thickness of the membrane bearing the nanopore. In such systems membrane thickness should be as thin as possible, ideally not thicker than the size of a nucleotide (˜0.3 nm). This requirement not only limits the choice of suitable materials to exotic candidates such as graphene monolayers for example, but also hampers the mechanical stability of the membrane construction. Poor stability is a severe problem in the practical applications of solid state nanopores thus far.

As an alternative to the vertical current readout with currents passing through the nanopores together with and in parallel to the DNA strands, ultrathin lateral electrodes split into two symmetric half-shells opposing each other around the nanopore have been investigated. When a nucleotide is passing through a nanopore in between the split two opposing half-shell electrodes on each side, the current between the lateral electrodes is modulated. Again, the thickness of the electrodes must be on the order of a nucleotide size in the sub-nm regime. Despite this sub-nm thickness, the detection zone for individual nucleotides is undesirably larger in the z-direction because electrical field-lines extend beyond the plane of the electrodes in both directions, yielding unsatisfying resolution performance. Nanostructures such as these also pose challenges in their technical realization. A benefit of such systems is that membranes can be made more stable, since the requirement for “ultrathin” exists only for the electrodes, while the carrying thin-film structure below can be designed thicker and thus more robust.

Nanochannels to guide DNA through, equipped with integrated comb electrodes (with dielectric nanogaps insulating the comb electrodes from each other) are another possible solution. This solution however comes at the cost of a highly complex manufacturing process. Nucleotide detection depends strongly on the distance and orientation of the DNA with respect to the nanogap electrodes. If redox molecules are used in the form of labels attached to certain types of nucleotides (to amplify electrical currents and to electrically discriminate different nucleotide types by different redox potentials of their attached redox labels through the comb electrodes), the distance and orientation of the DNA-attached redox molecule labels towards the comb electrodes is most critical for a successful readout. Orientation dependance leads to information loss in that only events that are in close proximity to the electrodes, meaning only events that are at the correct orientation, will be measured. More than 50% of potential readouts are expected to be lost due to this orientation dependance.

The present disclosure provides methods and systems for nanopore sequencing that overcome the aforementioned limitations and offer additional solutions.

Central to this disclosure is the discovery that redox-molecule labels attached to particular nucleotides yield high characteristic currents on the pA-scale when they pass through the center of two electrodes separated by a dielectric insulator of a few nm thickness only. Since redox molecule classes can be electrically distinguished from each other, depending on their redox potentials by the respective voltage and current curves, nucleotides can be distinguished electrically as well by the redox-labels attached to them. Current variations caused by a proximity effect via the redox molecules are ˜3 orders of magnitude larger than that usually observed with classical nanopores, an effect which has not been known outside of this research so far. The currents request close proximity and an exact centering of the redox molecule labels between the two metal electrodes in the z-direction, which focuses discovery of redox molecule labels to the symmetry plane between both electrodes. This yields a very narrow detection zone above and below the symmetry plane in the sub-nm regime. Neither the thickness of the metal electrodes nor the thickness of the dielectrics separating and electrically isolating them has an immediate negative effect on the resolution of the nucleotide detection concept, although there is a dependance on the thickness of the dielectrics. The latter should be designed as small as possible, however taking into account the requirement for sufficient stability towards electrical breakdown.

The systems and methods described herein implements the latter discoveries, mainly on the current characteristics, which represents the crux of the disclosed solutions. In at least an embodiment, a device may be provided. The device may include a layer stack. The layer stack may include a membrane comprising a first metal and a second metal separated by a dielectric insulating layer. The first metal, dielectric insulating layer, and second metal of the membrane together may comprise a metal-insulator-metal (MIM) electrode stack. The first and the second metal may be from 10-150 nm thick. The first metal and/or the second metal may comprise platinum or gold for example. The first metal may be the same as the second metal. Alternatively, the first metal may be different from the second metal. The dielectric insulating layer may comprise aluminum or silicon oxide for example. The dielectric insulating layer may be from 1 to 10 nm or from 2 to 7 nm, or from 3 to 5 nm thick. The total thickness of the membrane of the layer stack may be from about 50 to 400 nm, or from 100 to 300 nm. This may represent a stable mechanical construction which may be robust toward handling and processing loads.

Nanoholes may be fabricated through the membrane. Various technologies may be used to fabricate the nanoholes including but not limited to focused ion-beam etching, EUV lithography+plasma etching, ebeam-lithography+plasma etching, Xray or synchrotron lithography+plasma etching, or other alternatives for nanostructuring nanoholes. The nanohole diameter may be from about 1 to 10 nm, or about 2 to 7 nm, or about 3 to 5 nm. A diameter of 3 to 5 nm for example, may ensure close proximity of the through passing polynucleotide to the metal electrodes wherever the polynucleotide travels along.

The electrodes may surround the nanochannel in a rotation-symmetric arrangement. The nanogap would therefore surround the nanochannel as well. As a particular feature of the rotation-symmetric arrangement, the orientation of the polynucleotide or the attached redox-molecule labels at the time of passage is no longer critical. Whatever the orientation of the polynucleotide or the attached redox-molecule label may be, it will be identified properly because the electrodes surround the nanochannel in a rotation-symmetric arrangement. The discovery rate of redox-labeled nucleotides may therefore be close to 100%.

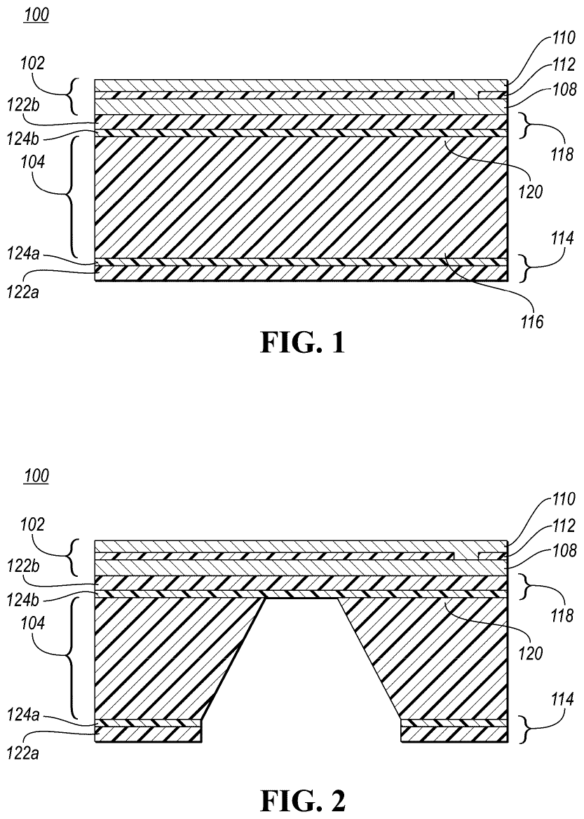

FIGS. 1-9 illustrate a fabrication process and a design realization according to at least some embodiments.

FIG. 1 illustrates a device 100 including a layer stack 102 on a silicon wafer 104. The layer stack may include a first metal 108 and a second metal 110 separated by a dielectric insulating layer 112. The first metal 108, dielectric insulating layer 112, and second metal 110 together may form a metal-insulator-metal electrode stack. The first metal 108 and the second metal 110 may be electrodes. The silicon wafer 104 may include a first stack 114 disposed on a first side 116 of the silicon wafer, and a second stack 118 disposed on a second side 120 of the silicon wafer. The first stack 114 and the second stack 118 may each include a first layer 122a, 122b (“122”) disposed on the silicon wafer 104 and a second layer 124a, 124b (“124”) disposed on the first layer 122. The first layer 122 may comprise SiO₂. The first layer 122 may be from about 30 to 150 nm, or about 40 to 100 nm, or about 50 nm thick. The second layer 124 may comprise Si₃N₄. The second layer 124 may be from about 70 to 420 nm, or about 100 to 300 nm, or about 140 nm thick. The first stack 114 and the second stack 118 may serve as an etching-mask and an etching-stop for through-wafer-etching from the backside. The through-wafer-etching from the backside may be performed in hot alkaline solutions including but not limited to potassium hydroxide (KOH) or tetramethyl ammonium hydroxide and water (TMAHW) for example, or by plasma etching through-wafer using deep reactive ion etching (e.g. “BOSCH-DRIE”).

FIG. 2 illustrates a cross section view of the layer stack 102 disposed on the silicon wafer 104 after backside etching through the wafer 104 according to an embodiment. KOH-etching of 100-wafers may be used as the etching technology, yielding the characteristic sidewall angles of 54.7°. In contrast to this, vertical sidewalls may be achieved by plasma etching.

FIG. 3 illustrates the removal of the etch-stop layer underneath the metal-oxide-metal electrode stack as well as of the backside masking layer, both as process options, to achieve a membrane thickness of 50-400 nm, preferably 100-300 nm. Removing the etch-stop and backside masking layer may be a useful step in the fabrication process but are not strictly required. A thickness within this range is suitable for etching a nanohole 126 through the layer stack 102 as the next step. The removal of SiO₂ and Si₃N₄ may be performed by a mixture of XeF₂ combined with SiF₂, SiF₃, etc. (unsaturated reaction products of Si and fluorine radicals from XeF₂), or by wet etchants like HF-solution or NH₄HF₂-solution.

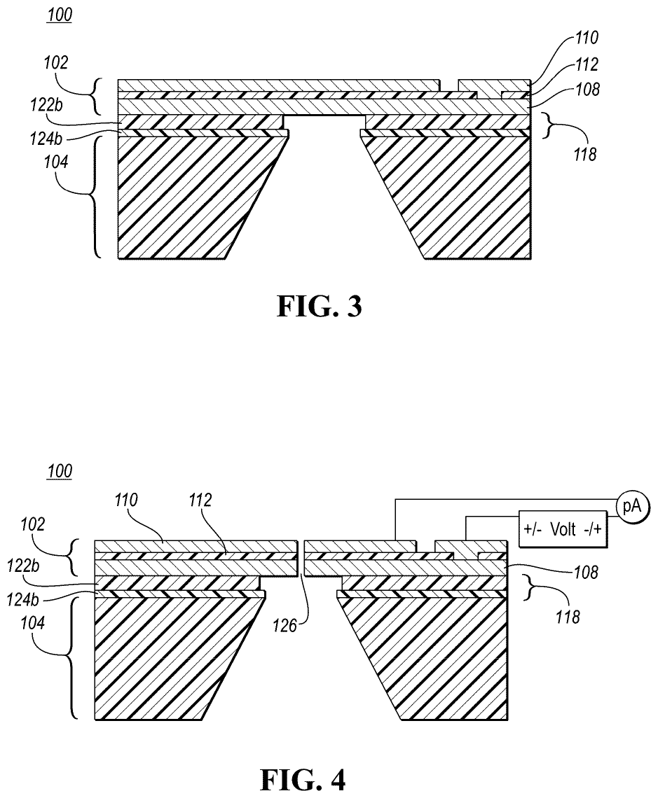

FIG. 4 shows a completed device 100 according to an embodiment. The device 100 includes a nanohole 126 drilled through the layer stack 102. The nanohole 126 may be drilled through the layer stack 102 by nanofabrication technologies including but not limited to focused ion-beam etching, or EUV lithography+plasma etching, or ebeam-lithography+plasma etching, or Xray or synchrotron lithography+plasma etching, as well as other alternatives for nanostructuring nanoholes. In addition, FIG. 4 depicts an electrical arrangement for current measurement and included voltage switching or ramping. For evaluation of different distinguishable redox molecule labels, the sampling voltage must be ramped through the different redox potentials and the respective currents measured synchronously.

It is recognized that this example represents a particular example of a design and fabrication method for a device 100. Other variations are contemplated which may yield a similar outcome and achieve the goals of this disclosure. The core of this disclosure is the implementation of a redox-molecule labeled polynucleotide strand analysis technique into a rotationally symmetric readout structure, which is free from the restrictions and drawbacks of previous solutions.

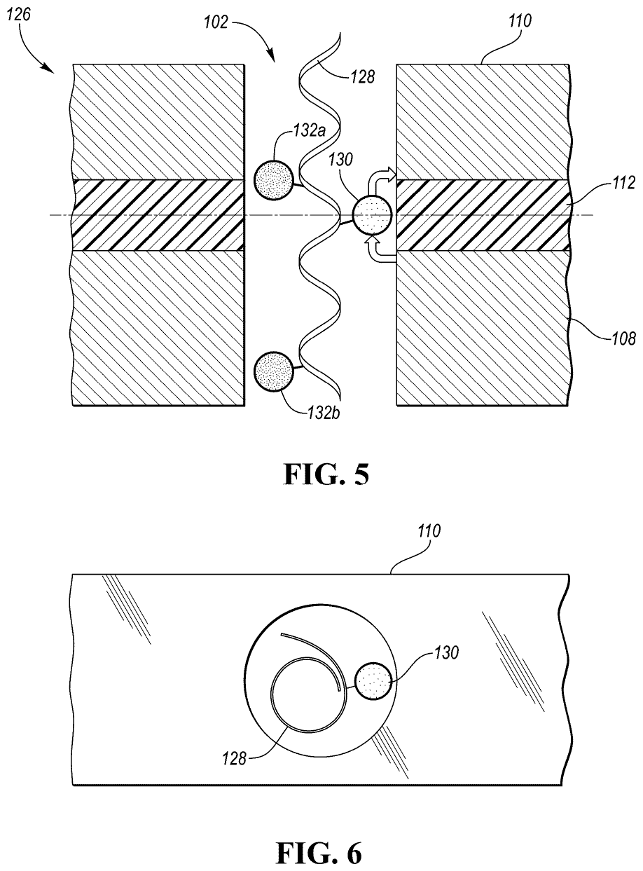

FIGS. 5 and 6 illustrate a cross-section view and a top view respectfully of a redox-labeled polynucleotide strand 128 inside a nanohole126 in-between the metal electrodes on the membrane according to an embodiment. Some or all of the nucleotides in the polynucleotide strand may have a redox molecule label (also referred to as an electroactive label) covalently bound to the nucleotide. At least two different types of labeled nucleotides must be present in the polynucleotide strand to determine the sequence of the strand. For example, referring to FIG. 5, a first redox molecule label 130 and a second redox molecule label 132a, 132b (“132”) represent electrically distinguishable redox labels, which yield different and well distinguishable currents at their characteristic and well-defined electrical potentials or voltages applied to the electrodes. Only the first redox molecule label 130 which is exactly centered along the z-direction with respect to the two-electrode-arrangement (in the symmetry plane between both electrodes) will yield a strong signal in the form of a large characteristic current occurring at the correct characteristic voltage levels (depending on the redox potential of the label). It is distinguishable from the second redox molecule label 132 by its individual redox potential corresponding to a particular voltage/current response when passing by the electrodes. Voltage sweeps or voltage switching, and current sampling may be performed continuously for sequential redox label discovery.

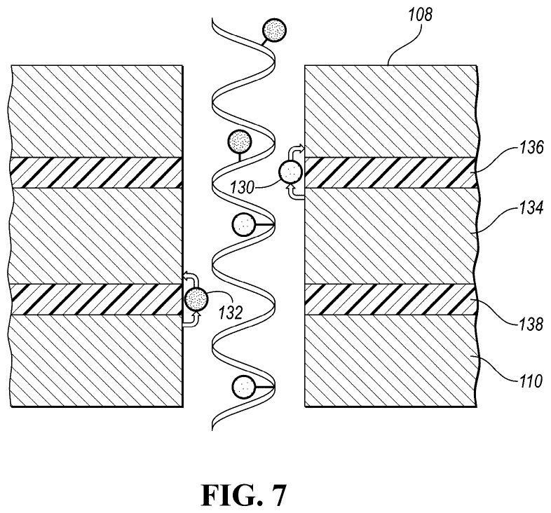

FIG. 7 illustrates a three-electrode arrangement, which enables detection of two distinguishable redox labels within the same nanohole. The first electrode 108 and a third electrode 134 may be separated by a dielectric insulating layer 136. The third electrode 134 is separated from the second electrode 110 by a second dielectric insulating layer 138. When electrode voltage V1 is different from V2, two electroactive labels with distinct redox characteristics can be detected while traversing a vertical nanohole. V1 can oxidize a first electroactive label 130 and V3 can reduce a first electroactive label 130. V2 can oxidize a second electroactive label 132 and V3 can reduce a first 130 and a second 132 electroactive label. Alternatively, V3 can be set up as an oxidizing electrode for both electroactive labels, while V1 can reduce a first electroactive label 130 and V2 can reduce a second electroactive label 132.

In another embodiment, V1=V2. The same electroactive label can be detected multiple times while traversing a vertical nanohole. V1 and V2 can oxidize an electroactive label and V3 can reduce an electroactive label.

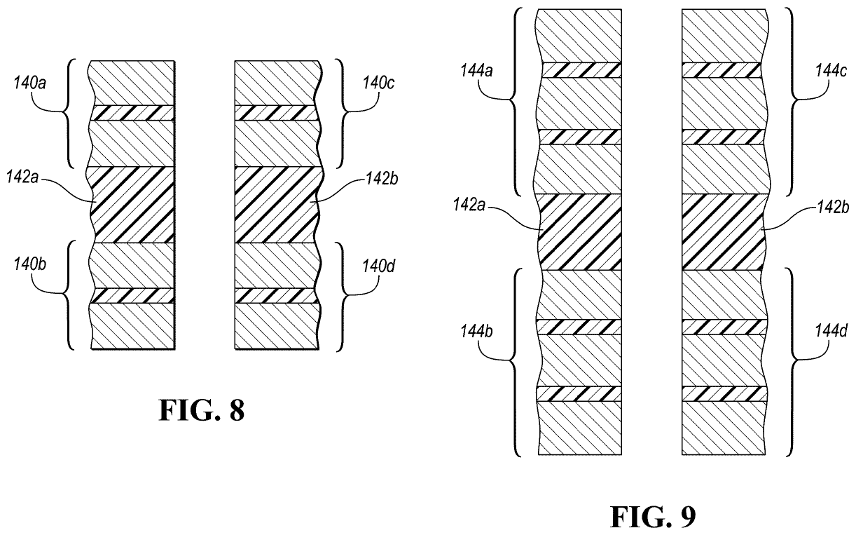

A pattern of two electrodes comprising a vertical nanogap 140a-140d (“140”) or of three electrodes comprising two vertical nanogaps 144a-144d (“144”) may be repeated within a nanohole multiple times. FIG. 8 illustrates an electrode arrangement that includes two electrodes separated by a nanogap 140. The nanogap may also be referred to as a dielectric insulating layer. The two electrodes separated by the nanogap 140 may also be referred to as a layer stack 140. Each pair of electrodes or layer stack 140 is separated from the next one by an insulating layer 142a, 142b (“142”). The pattern is repeated N times. In this way, a device according to an embodiment may include a first layer stack 140a including a first and second electrode separated by a dielectric insulating layer and a second layer stack 140b including a third electrode separated from a fourth electrode by a dielectric insulating layer. The second layer stack 140b may be separated from the first layer stack by an insulating layer 142a.

FIG. 9 represents an electrode arrangement that includes three electrodes separated by two identical nanogaps 144a-144d (“144”). Each group of electrodes 144 is separated from the next group by an insulating layer 142. Each group of electrodes may also be referred to as a layer stack 144. The pattern is repeated N times. In this way, a device according to an embodiment may include a first layer stack 144a including a first and third electrode separated by a dielectric insulating layer, and a second layer separated from the third electrode by an additional dielectric insulating layer. The device may also include a second layer stack 144b including a fourth electrode separated from a fifth electrode by a dielectric insulating layer and a sixth electrode separated from the fifth electrode by a dielectric insulating layer. The first layer stack 144a may be separated from the second layer stack 144b by an insulating layer 142a.

Polynucleotide strand or nucleotide transport through the nanopore may be driven by a second electrical potential across the nanohole in the z-direction, by separate (remote) electrodes above and below the membrane. This electrical potential can be adjusted freely to meet the desired polynucleotide strand pulling force and passage speed. Other ways to move the polynucleotide strand may include pressure driven, magnetic and enzyme mediated, mechanically mediated, temperature driven flow or a combination.

Examples of redox molecules that may be used as labels include metal-organic complexes, such as ferrocene and its derivatives, osmium and ruthenium complexes, conjugated organic molecules, such as tetrathiafulvalene, methylene blue, anthraquinone, phenothiazine, aminophenol, nitrophenol, erythrosine B, ATTO MB2, etc. The redox species must undergo a reversible oxidation-reduction reaction under applied electrical potential in order to enable the redox detection principle.

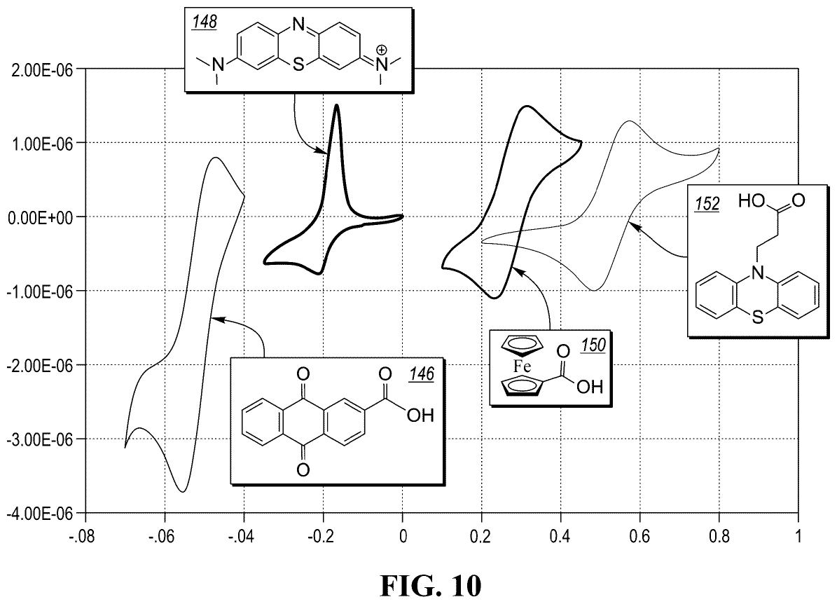

Below are selected examples of redox molecules that may be used with the systems and methods described in this disclosure. FIG. 10 illustrates cyclic voltammograms (CV) of electroactive labels. Anthraquinone carboxylic acid (green) 146, methylene blue (blue) 148, ferrocene carboxylic acid (orange) 150, and phenothiazine carboxylic acid (red) 152. The CVs are recorded in 10 mM Phosphate buffer solution containing 100 mM KCl and 10% DMSO. These molecules demonstrate electrochemically distinguishable voltage-current behavior and therefore may be used for polynucleotide strand sequencing when covalently attached to different types of nucleotides in a polynucleotide strand.

The labels may be used to provide a unique electronic signature for each type of nucleotide and to facilitate their identification. A system according to an embodiment may include the device 100 and a controller, a voltage supply and a current measurement unit, and a second voltage supply for the pull-voltage. To identify an incorporated nucleotide, a controller may apply voltages to the electrodes 108, 110 of the device 100 in the form of, for example, potentiometry, cyclic voltammetry, square wave voltammetry, or linear sweep voltammetry. The applied voltages may cover a pre-set range corresponding to redox reactions of the electroactive labels. The controller may measure the current (I) as a function of applied potential (V). The signature of each electroactive label may be determined prior to utilizing the electroactive label in a sequencing reaction. As each electroactive label has a unique I-V signature, this unique signature may be used to identify the label, and therefore the nucleotide carrying the label. Voltages may be swept on both electrodes on a nanogap sensor independently (applying reducing voltages to the first electrode and oxidizing voltages to the second electrode). Alternatively, a wave form that covers both reducing and oxidizing potentials may be applied to one or both electrodes. The controller may also set and adjust the “pull-through” voltage from the pull-voltage source, causing the polynucleotide strands to pass the nanopore at a pre-defined speed.

The system may further include a modulator and a demodulator. The modulator applies an AC signal to the nanohole. The synchronized demodulator may also be referred to as a mixer. Measured currents (I) may be synchronously demodulated (phase sensitive synchronous demodulator by mixing and subsequent low-pass-filtering) with respect to the applied potential voltage (V) or with respect to any applied potential voltage (V) as a reference signal. Other contributions to the current (I) which are not synchronous in phase and frequency with the voltage sweep are thus efficiently discriminated. The demodulator/mixer may act as a spectral filter with phase-sensitivity and narrow bandwidth corresponding to the bandwidth of the low-pass-filter. The approach of periodic voltage variation combined with synchronous demodulation of current variation (I) may improve the signal-to-noise-ratio and suppression of irrelevant current contributions. In addition, by using different frequencies for different electrode pairs, cross-talk may be reduced.

The major benefits of the systems and methods described herein are the mechanical robustness of the device, large currents in the pA-regime via the redox molecules when the redox-labeled nucleotides are passing through the detection plane, which is the plane of symmetry between the first and second metal electrodes, and the discovery of practically every redox label due to the rotational symmetry of the detector electrode arrangement around the nanoholes or nanopores which makes detection independent from label or nucleotide orientation. In addition, detection voltages/currents and driving voltages (pulling forces) and thus polynucleotide strand passage speed are decoupled and may be adjusted independently from each other.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A system for sequencing a polynucleotide strand comprising:
a device including:
a layer stack on a silicon wafer, the layer stack comprising a first electrode and a second electrode separated by a dielectric insulating layer;
a nanohole drilled through the layer stack so that the first and second electrodes are rotationally arranged around the nanohole, wherein the nanohole is sized to receive a polynucleotide strand with at least one nucleotide and at least one electroactive label; and
a controller configured to:
apply an electrical signal to the first and second electrodes;
measure a current (I) as a function of an applied potential (V) when an electroactive label is guided through the nanohole of the device; and
adjust an electrical voltage to pull the polynucleotide strand through the nanohole at a predetermined speed.

2. The system of claim 1, further comprising a mixer configured to synchronously demodulate the measured current with respect to a modulation of a voltage level.

3. The system of claim 1, wherein the layer stack is from 50 to 400 nm thick.

4. The system of claim 1, wherein the layer stack is from 100 to 300 nm thick.

5. The system of claim 1, wherein the nanohole has a diameter of 1 to 10 nm.

6. The system of claim 1, wherein the first and the second electrode each comprise a metal to 150 nm thick.

7. The system of claim 1, wherein the dielectric insulating layer is from 1 to 10 nm thick.

8. The system of claim 1, wherein the device further comprises a second layer stack including a third electrode separated from a fourth electrode by a dielectric insulating layer, wherein the second layer stack is separated from the layer stack by an insulating layer.

9. The system of claim 1, wherein the layer stack further comprises a third electrode separated from the second electrode by a dielectric insulating layer.

10. The system of claim 9, wherein a second layer stack including a fourth electrode separated from a fifth electrode by a dielectric insulating layer and a sixth electrode separated from the fifth electrode by a dielectric insulating layer is separated from the layer stack by an insulating layer.

11. A method for sequencing a polynucleotide strand comprising:
providing a sample containing a polynucleotide strand with a first nucleotide having a first electroactive label and a second nucleotide having a second electroactive label, the second electroactive label being distinguishable from the first electroactive label, to a device including a layer stack on a silicon wafer, the layer stack comprising a first electrode and a second electrode separated by a dielectric insulating layer, and a nanohole drilled through the layer stack so that the first and second electrodes are rotationally arranged around the nanohole;
applying an electrical signal to the first and second electrodes; and
measuring a current (I) as a function of an applied potential (V) when the first and the second electroactive labels are present in the nanohole.

12. The method of claim 11, further comprising synchronously demodulating the measured currents with respect to a modulation of a voltage level.

13. The method of claim 11, wherein the layer stack further comprises a third electrode separated from the second electrode by a dielectric insulating layer.

14. The method of claim 11, wherein the layer stack is from 50-400 nm thick.

15. The method of claim 11, wherein the layer stack is from 100 to 300 nm thick.

16. The method of claim 11, further comprising applying a second electrical signal across the nanopore in the z-direction to control passage of the polynucleotide strand through the nanopore.

17. A method for forming a system for sequencing a polynucleotide strand comprising:
assembling a base wafer by forming an etching mask on a backside of a silicon wafer, an etching stop on an opposite side of the silicon wafer, and a layer stack comprising a first electrode and a second electrode separated by a dielectric insulating layer on the etching stop;
backside etching through the base wafer;
removing the backside etching mask;
opening a front side window;
forming a nanohole through the layer stack, wherein the first and second electrodes are rotationally arranged around the nanohole to form a device; and
configuring a controller to apply an electrical signal to the first and second electrodes, and to measure a current (I) as a function of an applied potential (V) when an electroactive label is present in the nanohole.

18. The method of claim 17, wherein the layer stack is from 50 to 400 nm thick.

19. The method of claim 17, wherein the layer stack is from 100 to 300 nm thick.

20. The method of claim 17, wherein the nanohole has a diameter of 1 to 10 nm.

21. The method of claim 17, further comprising forming a second layer stack separated from the layer stack by an insulating layer.

22. The method of claim 17, further comprising forming a layer stack including a third electrode separated from the second electrode by a dielectric insulating layer.

23. The method of claim 22, further comprising forming a second layer stack separated from the layer stack by an insulating layer.
